(19) United States
(12) Patent Application Publication
(10) Pub. No.: US 2011/0238044 A1
(43) Pub. Date: Sep. 29, 2011

(54) SURGICAL INSTRUMENT, HANDLE FOR A SURGICAL INSTRUMENT AND SURGICAL INSTRUMENT SYSTEM

(75) Inventors: David Main, West Yorkshire (GB); Michael White, West Yorkshire (GB)

(73) Assignee: Surgical Innovations Limited, Leeds, Yorkshire (GB)

(21) Appl. No.: 13/131,766

(22) PCT Filed: Dec. 4, 2009
(86) PCT No.: PCT/GB2009/051651

§ 371 Date: May 27, 2011

(30) Foreign Application Priority Data
Dec. 5, 2008 (GB) 0822225.9

(51) Int. Cl. A61B 17/00

(52) U.S. Cl. 606/1

(57) ABSTRACT

A surgical instrument (4), a handle (2) for use with the surgical instrument and a surgical instrument system are described. The surgical instrument and handle comprise complementary features to ensure a secure connection between the instrument and handle. hi one embodiment the instrument comprises an outer shaft (12), an inner shaft (14) and an operative portion. The outer shaft comprises opposed flat surfaces (16) allowing it to be engaged by the handle on the flat surfaces. The inner shaft comprises a latch surface (26) at its proximal end for engaging a latch mechanism in the handle. In other embodiments a radial bore (18) is provided in the outer shaft of the instrument. This can be engaged by an elongated member in the handle.

The present invention relates to a surgical instrument for use in minimally invasive surgery. The present invention also relates to a handle for the surgical instrument and a system comprising the surgical instrument and the handle.

Minimally invasive surgical techniques are known. In such techniques a surgeon typically makes a small incision into which a specialised instrument is inserted and then manipulated by the surgeon. Many instruments for use with minimally invasive surgery have been proposed. One type of instrument comprises an elongated shaft with an operative part disposed at one end and a handle at the other. The handle comprises two parts that can move relative to each other and which are attached, directly or indirectly, to the operative part. For example the operative part may be surgical scissors and relative movement of the two parts of the handle may open and close the scissors.

Instruments have been proposed in which the shaft and operative part are releasably attached to the handle. This allows the use of the same handle with different operative parts. It also allows the shaft and operative part to be made disposable or to be sterilised separately from the handle.

There are two main criteria that a releasable attachment system for a minimally invasive surgical instrument must meet. Firstly, the connection between the handle and the instrument must ensure that the risk of accidental release of the surgical instrument from the handle is minimised. Secondly, the connection must ensure that any rotational movement of the handle about the axis of the elongated shaft is transmitted to the surgical instrument.

An example of a surgical instrument in which the shaft and operative part are releasably attached to a handle is discussed in U.S. Pat. No. 5,630,832. A pair of mutually articulated grip shanks are provided on a handle. A tube is secured on one of the shanks and a rod-like actuator is fitted to move longitudinally in the tube and articulated on the other shank. A realeasable connection between one shank and the tube is provided through a spherical locking member that can move radially and which engages a recess on the tube. A releasable connection between the actuator and the other shank is provided by a swivel lever in the handle with a blind hole bore that engages a spherical end of the actuator.

The use of spherical locking members as the primary means of securing the tube in U.S. Pat. No. 5,630,832 create some disadvantages. A single spherical locking member provides relatively poor locking between the handle and the tube, so that three or more may be required, further increasing the complexity. The tube must have curved recesses corresponding to the shape of the spherical member machined, increasing the complexity of manufacture.

It would be desirable to provide a releasable attachment system for a surgical instrument with a relatively simple yet secure attachment mechanism.

Accordingly, the present invention provides a surgical instrument with an outer shaft having a pair of opposed flat surfaces which are substantially parallel to each other. The flat surfaces can be engaged by a locking member in the handle to releasably connect the handle to the shaft of the surgical instrument.

Alternatively or in addition, the present invention provides a surgical instrument with an outer shaft having a radial bore. The radial bore can be engaged by a movable elongated member in the handle to releasably connect the handle to the shaft of the instrument.

The use of flat surfaces and a radial bore can be combined if required. All of these systems can be manufactured simply and provide secure locking

According to a first aspect of the present invention, there is provided a surgical instrument for use in minimally invasive surgery. The instrument comprises:
an outer shaft having proximal and distal ends, a longitudinal axis and a through bore extending from the proximal end to the distal end;
an inner shaft having proximal and distal ends positioned within the through bore and extending proximally beyond the proximal end of the outer shaft; and
an operative portion comprising a first part attached to the distal end of the outer shaft and a second part attached to the distal end of the inner shaft; wherein
a portion of the outer shaft towards the proximal end of the outer shaft has an external surface comprising two opposed flat surfaces which are substantially parallel to each other; and
wherein the proximal end of the inner shaft comprises an end portion with a maximum external dimension in a direction perpendicular to the longitudinal axis greater than the maximum external dimension of an adjoining portion of the inner shaft, and wherein a latch surface substantially perpendicular to the longitudinal axis extends between the end portion and the adjoining portion.

The latch surface may be engaged by a latch in a handle for the surgical instrument. The end portion may be curved such that the external dimension of the end portion perpendicular to the longitudinal axis reduces towards the proximal end. More preferably the end portion is at least partially spherical. Providing a curved surface on the end portion enables a latch in the handle to be engaged smoothly when the surgical instrument is inserted into the handle.

The reference to “proximal” ends throughout this document refers to the end of the surgical instrument closest to a user of the instrument. The reference to “distal” ends throughout this document refers to the end of the instrument furthest from a user of the instrument.

The operative portion can be any surgical instrument in which relative movement of two parts is required to operate the instrument. The first and second parts of the operative portion can be directly or indirectly attached to the outer and inner shaft. Examples of the operative portion include forceps or scissors, amongst others. The outer shaft may be a tube. The opposed flat surfaces of the outer shaft enable the outer shaft to be secured in the handle against rotation about the longitudinal axis. The flat surfaces can be formed easily during manufacture.

In one embodiment, the outer shaft may have a greatest external dimension in the direction perpendicular to the two opposed flat surfaces, and wherein the greatest external dimension is greater than the distance between the two opposed flat surfaces. This means that the distance between the two flat surfaces is narrower than a portion of the outer shaft, so that by engaging the two flat surfaces the outer shaft is also secured against removal from the handle along the longitudinal axis.

The outer shaft may be substantially cylindrical and in that case the distance between the two opposed flat surfaces may be less than the external diameter of the outer shaft.

In some embodiments, the outer shaft further comprises a radial bore extending through the outer shaft in a direction substantially perpendicular to the longitudinal axis. This bore can be engaged by a movable elongated member in a handle for the instrument, providing an additional means of securing the shaft against rotation relative to the handle and accidental removal from the handle.

The inner shaft may comprise a section of reduced radial height adjacent to the radial bore. The section of reduced radial height may have a substantially planar surface. By providing this portion of reduced radial height the elongated member may extend completely through the radial bore so that an end of the elongated member is located in the space between the outer shaft and the section of reduced radial height. The elongated member can then prevent rotation of the inner shaft relative to the outer shaft about the longitudinal axis. This is particularly beneficial when the surgical instrument is reusable. It is common to at least partially disassemble a reusable surgical instrument for cleaning and sterilisation between uses. In some embodiments, the surgical instrument may be disassembled by rotating the inner shaft relative to the outer shaft to disconnect a threaded connection or a bayonet connection. If this relative rotation is prevented the instrument is prevented from inadvertent disassembly during use.

The radial bore may be located between the two opposed flat surfaces. The direction of the radial bore may be substantially parallel to the two opposed flat surfaces, in which case the radial bore is preferably located halfway between the two opposed flat surfaces.

According to a further aspect of the invention, there is provided a handle for use with a minimally invasive surgical instrument. The handle comprises:
a first handle part comprising a substantially cylindrical bore having a longitudinal axis for receiving a surgical instrument;
a second handle part attached to the first handle part for pivotal movement relative to the first handle part; and wherein
the first handle part further comprises a locking member extending through the substantially cylindrical bore that can be moved between first and second positions in a direction perpendicular to the longitudinal axis, wherein the locking member delimits an opening having a first portion with dimensions the same as or larger than the diameter of the substantially cylindrical bore and an adjoining second portion with at least one dimension smaller than the diameter of the substantially cylindrical bore, wherein in the first position the first portion is aligned with the substantially cylindrical bore and in the second position the second portion is aligned with the substantially cylindrical bore, the first handle part further comprises a latch mechanism at the end of the substantially cylindrical bore.

This handle can be used with a surgical instrument with two opposed flat surfaces on its outer shaft. The second portion of the locking member engages the two opposed flat surfaces in the second position. As the locking member has to be moved to lock, a user can tell at a glance whether a surgical instrument has been locked in place in the handle.

The latch mechanism may engage a latch surface formed at the end of the inner shaft of the surgical instrument to secure inner shaft to the handle.

The latch mechanism may comprise a latch member movable between first and second positions and wherein the latch member is biased in the first position by a resilient means, such that movement of the latch member into the second position is against a force exerted by the resilient means. The resilient means is a preferably a spring but can be other resilient material, such as an elastomer. The second position is preferably the position in which the latch member retains an inserted surgical instrument in the handle. In this way the force provided by the resilient means acts to retain the surgical instrument in the handle.

The latch member may comprise a surface angled relative to the longitudinal axis. This can interact with a curved surface at the end of the inner shaft of a surgical instrument so that when a surgical instrument is inserted, the latch is urged downwards smoothly against the force of the resilient means.

The latch mechanism may further comprise a release lever for moving the latch member into the second position. This enables a user to release the latch when required.

In one embodiment the locking member may further comprise an elongated member extending partially into the second portion of the opening in a direction perpendicular to the longitudinal axis. This enables the handle to engage a radial bore in the outer shaft of the surgical instrument, as well as the two opposed flat surfaces, for a more secure attachment.

In yet further aspects of the invention, the surgical instruments and handles of the above described aspects can be combined to provide a surgical instrument system.

According to another aspect of the invention, there is provided a surgical instrument system for use in minimally invasive surgery, wherein the system comprises:
a surgical instrument comprising:
   an outer shaft having proximal and distal ends, a longitudinal axis, and a through bore extending from the proximal end to the distal end;
   an inner shaft having proximal and distal ends positioned within the through bore and extending proximally beyond the proximal end of the outer shaft; and
   an operative portion comprising a first part attached to the distal end of the outer shaft and a second part attached to the distal end of the inner shaft; wherein
   a portion of the outer shaft towards the proximal end of the outer shaft has an external surface comprising two opposed flat surfaces which are substantially parallel to each other; and
a handle comprising:
   a first handle part comprising a substantially cylindrical bore for receiving a part of the outer shaft and the inner shaft;
   a second handle part attached to the first handle part for pivotal movement relative to the first handle part; and wherein
   the first handle part further comprises a locking member extending through the substantially cylindrical bore that can be moved between first and second positions in a direction perpendicular to the longitudinal axis, wherein the locking member delimits an opening having a first portion with dimensions the same as or larger than the diameter of the substantially cylindrical bore and an adjoining second portion with a dimension corresponding the distance between the two opposed flat surfaces, wherein in the first position the first portion is aligned with the substantially cylindrical bore and in the second position the second portion is aligned with the substantially cylindrical bore such that when the proximal end of the surgical instrument is inserted into the substantially cylindrical bore and the locking member is moved into the second position, the two opposed flat surfaces are engaged by the second portion,
wherein the proximal end of the inner shaft of the surgical instrument may comprise an end portion with a maximum external dimension in a direction perpendicular to the longitudinal axis greater than the maximum external dimension of an adjoining portion of the inner shaft, wherein a latch surface substantially perpendicular to the longitudinal axis extends between the end portion and the adjoining portion; and wherein the first handle part further comprises a latch mechanism at the end of the substantially cylindrical bore for engaging the latch surface.

The outer shaft of the surgical instrument may further comprise a radial bore extending through the outer shaft in a direction substantially perpendicular to the longitudinal axis, and wherein the locking member of the handle further comprises an elongated member extending partially into the second portion of the opening in a direction perpendicular to the longitudinal axis; such that when the proximal end of the surgical instrument is inserted in the substantially cylindrical bore and the locking member is moved into the second position, the elongated member engages the radial bore.

According to another aspect of the present invention, there is provided a surgical instrument for use in minimally invasive surgery. The instrument comprises:
an outer shaft having proximal and distal ends, a longitudinal axis and a through bore extending from the proximal end to the distal end;
an inner shaft having proximal and distal ends positioned within the through bore and extending proximally from the proximal end of the outer shaft; and
an operative portion comprising a first part attached to the distal end of the outer shaft and a second part attached to the distal end of the inner shaft; wherein
the outer shaft further comprises a radial bore extending through the outer shaft in a direction substantially perpendicular to the longitudinal axis located towards the proximal end of the outer shaft.

The radial bore can be engaged by a movable elongated member in the handle, securing the shaft against rotation relative to the handle and accidental removal from the handle.

The inner shaft may comprise a section of reduced radial height adjacent to the radial bore, which may have a substantially planar surface. This can enable an elongated member in the handle to prevent relative rotation between the inner and outer shafts along their longitudinal axis, to prevent inadvertent unscrewing of a reusable instrument.

According a still further aspect of the present invention, there is provided a handle for use with a minimally invasive surgical instrument. The handle comprises:
a first handle part comprising a substantially cylindrical bore having a longitudinal axis for receiving a surgical instrument;
a second handle part attached to the first handle part for pivotal movement relative to the first handle part; and wherein
the first handle part further comprises a locking member that can be moved between first and second positions in a direction perpendicular to the longitudinal axis, wherein the locking member comprises an elongated member that extends in a direction perpendicular to the longitudinal axis, such that in the first position an end of the elongated member is located further from the longitudinal axis than the diameter of the substantially cylindrical bore, and in the second position the end of the elongated member is located closer to the longitudinal axis than the diameter of the substantially cylindrical bore.

The elongated member can engage a radial bore formed in the outer shaft of a surgical instrument to secure the surgical instrument to the handle. The distance of the elongated member from the longitudinal axis may be chosen so that in the first position the distance is greater than the diameter of the outer shaft of the surgical instrument, so that there is no obstruction to its insertion. In the second position, the distance may be chosen so that the elongated member engages the radial bore formed in an inserted surgical instrument.

The locking member may extend through the substantially cylindrical bore and delimit an opening which is aligned with the substantially cylindrical bore, with the elongated member extending partially into the opening.

According to another aspect of the present invention, there is provided a surgical instrument system for use in minimally invasive surgery, wherein the system comprises:
a surgical instrument comprising:
   an outer shaft having proximal and distal ends, a longitudinal axis and a through bore extending from the proximal end to the distal end;
   an inner shaft having proximal and distal ends positioned within the through bore and extending proximally from the proximal end of the outer shaft; and
   an operative portion comprising a first part attached to the distal end of the outer shaft and a second part attached to the distal end of the inner shaft; wherein
   the outer shaft further comprises a radial bore extending through the outer shaft in a direction substantially perpendicular to the longitudinal axis and located towards the proximal end of the outer shaft; and
a handle comprising:
   a first handle part comprising a substantially cylindrical bore having a longitudinal axis for receiving the surgical instrument;
   a second handle part attached to the first handle part for pivotal movement relative to the first handle part; and wherein
   the first handle part further comprises a locking member that can be moved between first and second positions in a direction perpendicular to the longitudinal axis, and wherein the locking member comprises an elongated member that extends in a direction perpendicular to the longitudinal axis, such that in the first position an end of the elongated member is located further from the longitudinal axis than the diameter of the substantially cylindrical bore, and in the second position the end of the elongated member is located closer to the longitudinal axis than the diameter of the substantially cylindrical bore; such that, when the proximal end of the surgical instrument is inserted into substantially cylindrical bore and the locking member is moved to the second position, the elongated member engages the radial bore.

Embodiments of the invention will now be described by way of example with reference to the accompanying drawings, in which:

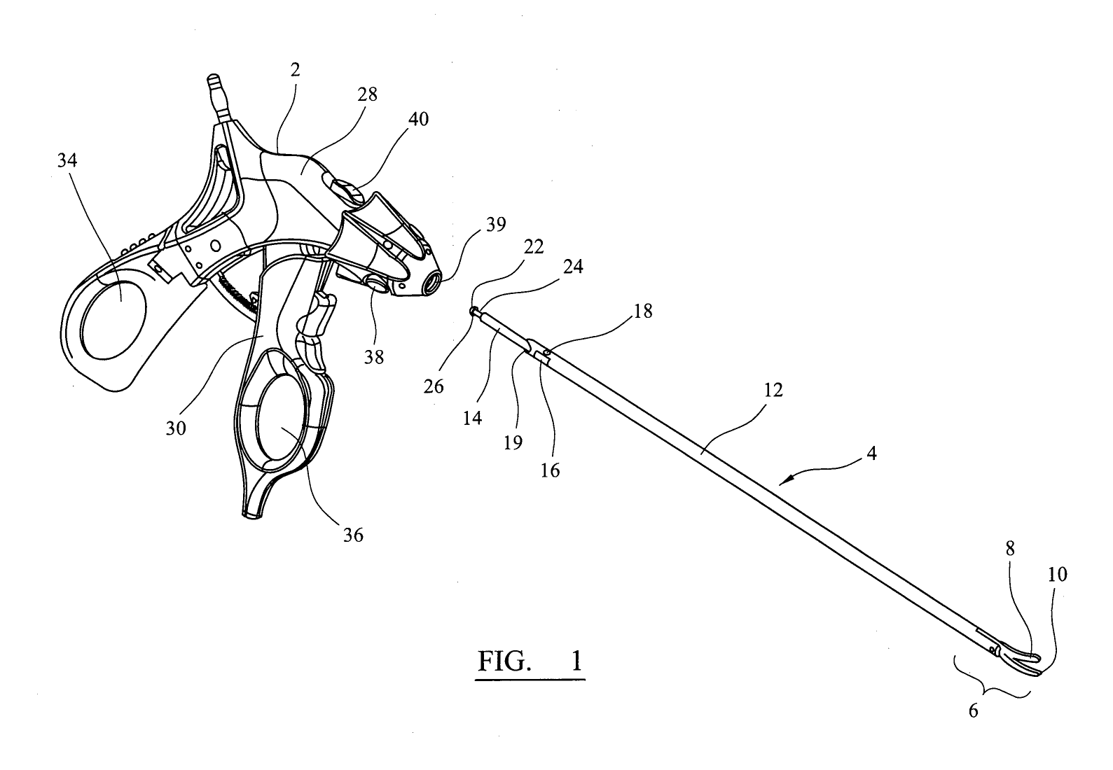

FIG. 1 depicts a perspective view of a first embodiment of the present invention.

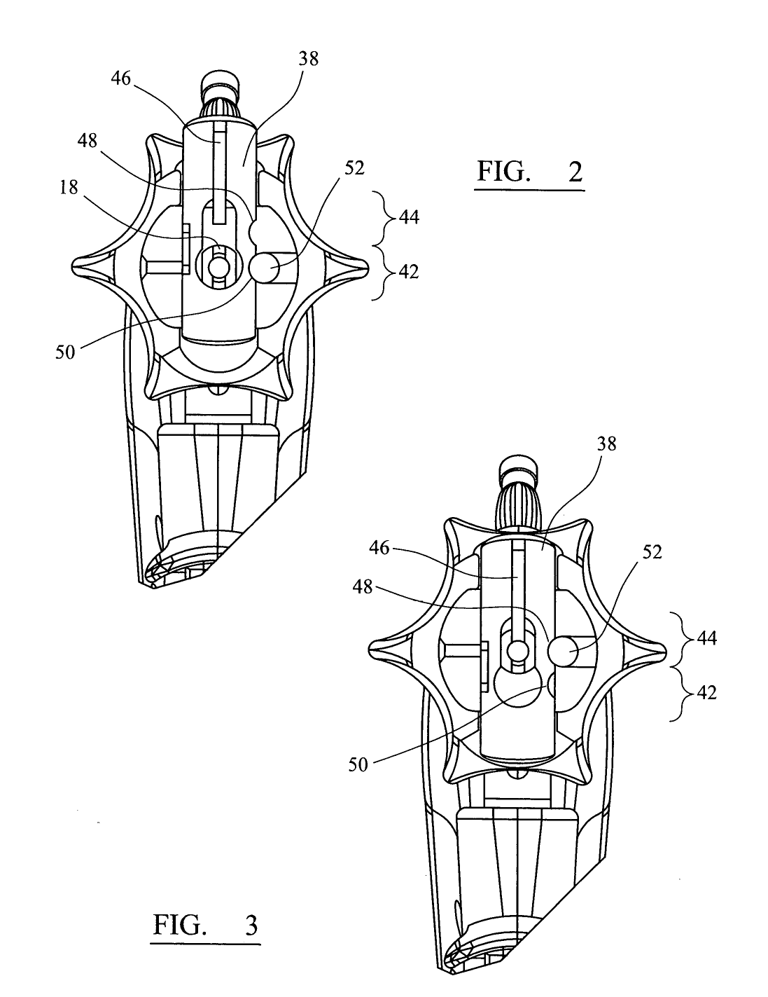

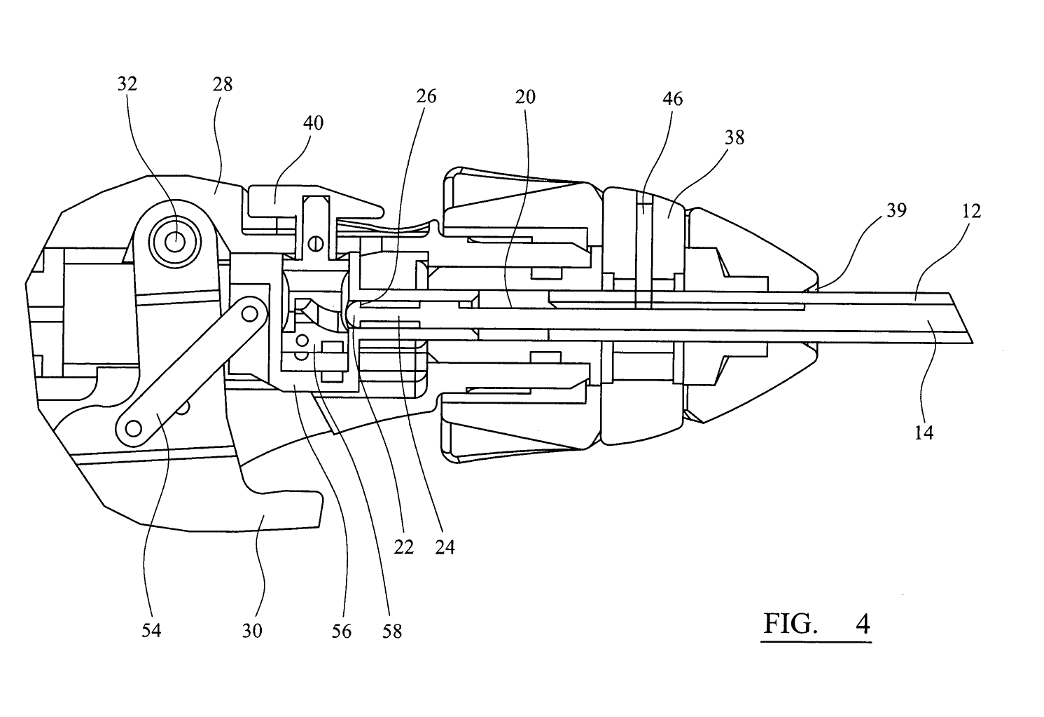

FIGS. 2 and 3 depict cross sections showing the locking of the handle onto the outer shaft of a surgical instrument in the embodiment of FIG. 1;

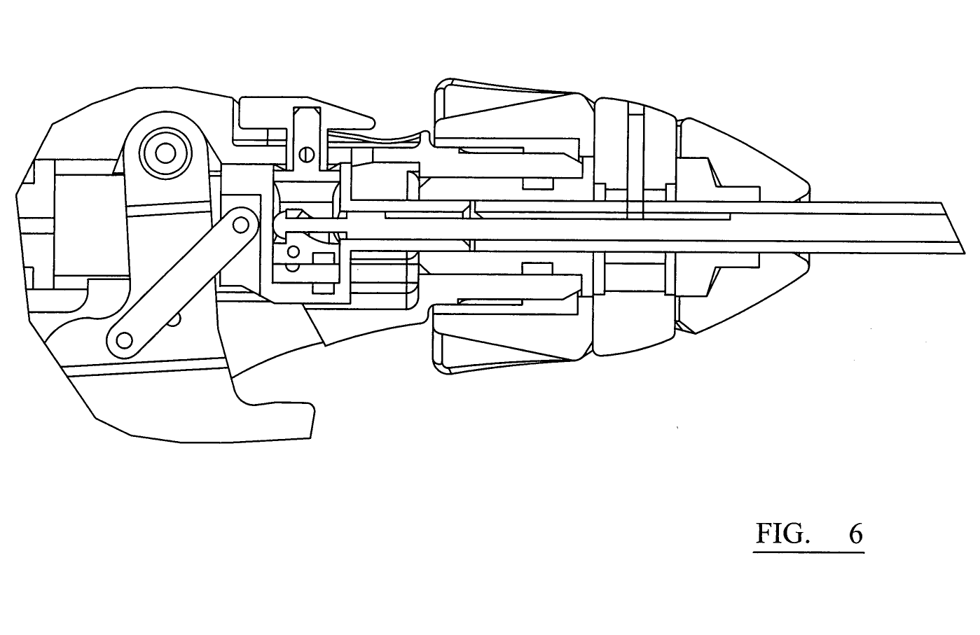

FIGS. 4-6 depict various stages in the latching of the handle onto the inner shaft of a surgical instrument in the embodiment of FIG. 1;

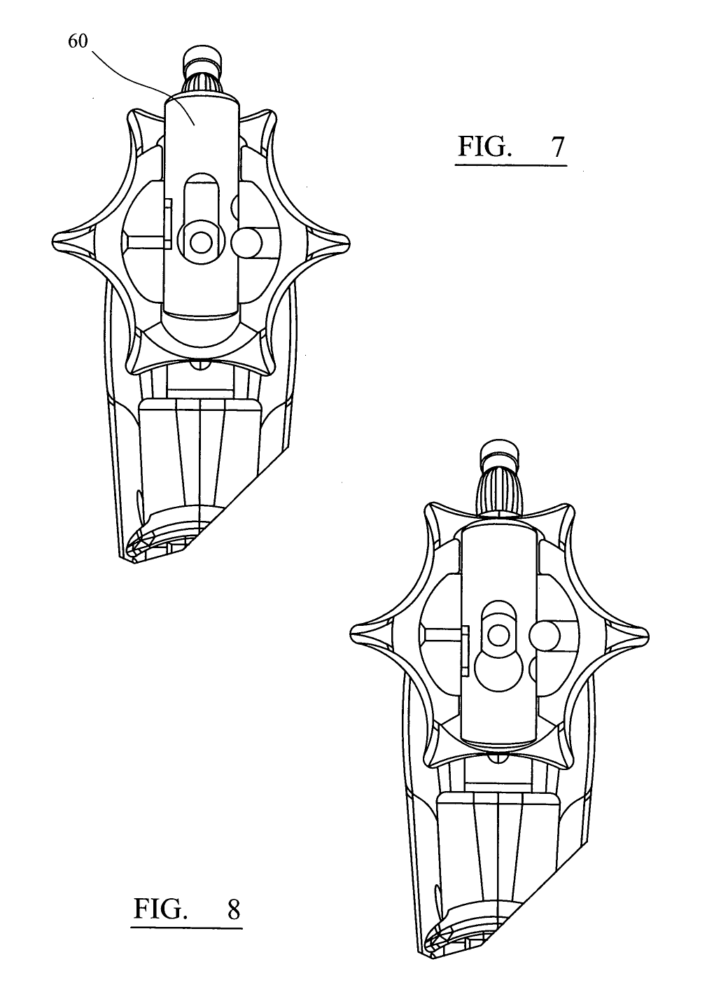

FIGS. 7 and 8 depict cross sections showing the locking of the handle onto the outer shaft of a second embodiment of the present invention; and

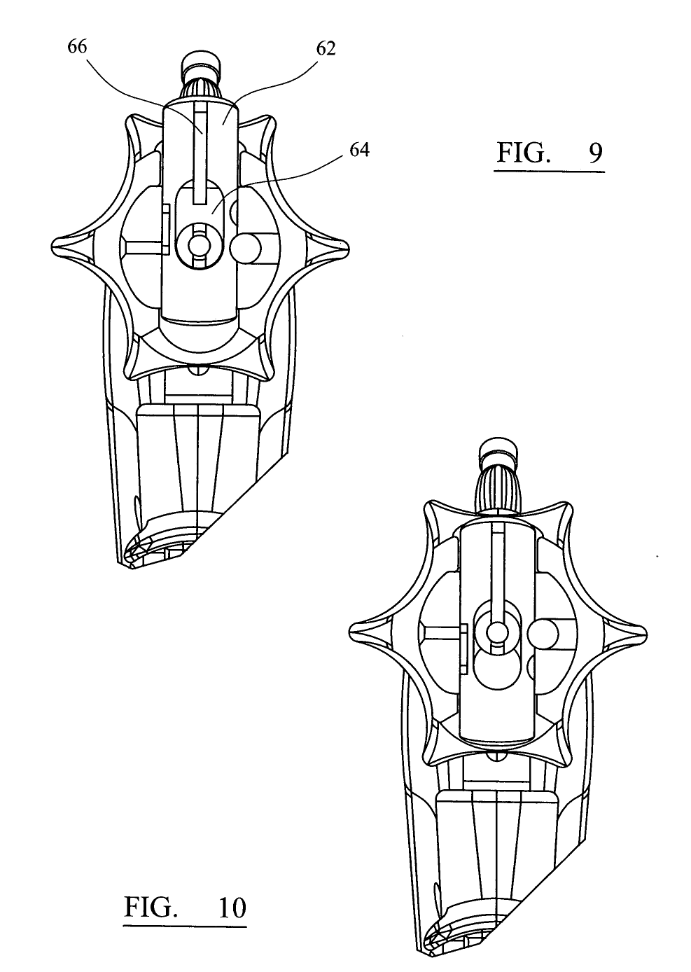

FIGS. 9 and 10 depict cross sections showing the locking of the handle onto the outer shaft of a third embodiment of the present invention.

FIG. 1 depicts a perspective view of a surgical instrument system according to a first embodiment of the present invention. The surgical instrument system comprises a handle 2 and a surgical instrument 4 that can be inserted into the handle 2. The surgical instrument system is intended for use in minimally invasive surgery. As such the surgical instrument comprises an operative portion 6 which in this case are surgical scissors. The scissors 6 comprise two main parts 8, 10 that can pivot relative to each other between open and closed positions. A first part 8 of the scissors 6 is attached to the distal end of an outer shaft 12, which is preferably in the form of a tube with a longitudinal axis so that a bore runs along the length of the outer shaft 12 from the proximal end to the distal end. An inner shaft 14 is positioned within the bore of the outer shaft 12. The distal end of the outer shaft 12 is attached to the second part 10 of the scissors 6.

The outer shaft 12 has two parallel, flat surfaces 16 formed opposite each other towards its proximal end. A radial bore 18 is formed midway between the two flat surfaces and perpendicular to the longitudinal axis of the outer shaft 12. Indentations 19 are formed at the proximal end of the outer shaft 12.

The inner shaft 14 extends beyond the proximal end of the outer shaft 12. In this embodiment the inner shaft 14 has a substantially circular cross section along it's length. At the proximal end of the inner shaft 14, this circular cross section is modified. A portion 20 of the inner shaft under the radial bore 18 and extending for some distance either side, for example 10 to 20 mm either side, is flattened so that the radial height of the inner shaft 14 under the radial bore 18 is lower (The portion 20 is not visible in FIG. 1, but can be seen in the cross section of FIG. 4). The proximal end of the inner shaft 14 terminates in a hemispherical end 22. Adjoining the hemispherical end 22, a portion 24 having a reduced diameter is formed. The transition from the hemispherical end 22 to the portion 24 having a reduced diameter is preferably a “step” change so that a latching surface 26 is formed, which is substantially perpendicular to the longitudinal axis.

The handle 2 comprises a first handle part 28 and a second handle part 30 which are connected for pivotal movement relative to each other by a rotational joint 32 (not shown, see the cross section of FIG. 4). Both the first and second handle parts 28, 30 include an opening 34, 36 in their lower portion for receiving a user's fingers or thumb. Preferably the opening 34 in the first handle part 28 is for receiving a user's thumb and the opening 36 in the second handle part 30 is for receiving one or more of the user's fingers.

The handle 2 also comprises a locking member 38 and a latch release member 40 in the first handle part 28. A substantially cylindrical bore 39 is formed at the distal end of the first handle part 28 for receiving the surgical instrument 4.

The construction of the locking member 38 will now be described in more detail with reference to the cross section of FIG. 2. The locking member 38 is received in corresponding opening in the first handle part 28 so that it can move between a first position (depicted in FIG. 2) and a second position (depicted in FIG. 3) in a direction perpendicular to the axis of the substantially cylindrical bore 39. The locking member has an opening formed through it that is aligned with the substantially cylindrical bore 39 and comprising a first portion 42 and a second portion 44. The first portion 42 has a generally circular cross section which is the same diameter or larger than the substantially cylindrical bore 39. The second portion 44 has a narrower cross section then the first portion 42 and comprises two parallel flat surfaces positioned slightly further apart then the flat surfaces 16 on the outer shaft 12.

An elongated member 46 is also provided in the locking member 38, and is fixed so that it cannot move relative to the locking member 38. The elongated member is preferably in the form of a cylinder and one end extends partially into the second portion of the opening 44.

An external surface of the elongated member comprises two indentations 48, 50. The first handle part includes a recess for receiving a spherical member, preferably a ball bearing 52. The ball bearing 52 is urged into one of the indentations 48, 50 by a resilient biasing means (not shown) which is preferably a spring. The first indentation 50 is positioned such that when it is engaged by the ball bearing 52 the locking member 38 is in the first position. The second indentation 48 is positioned such that when it is engaged by the ball bearing 52 the locking member 38 is in the second position. The use of ball bearing 52 provides positive feedback to the user of movement between the first and second positions and also acts to restrict accidental movement of the locking member from one position to another.

FIGS. 4 to 6 depict a cross section of the handle 2 along the longitudinal axis of the substantially cylindrical bore 39.

The rotational connection 32 between the first handle part and the second handle part 30 can be seen in more detail in FIG. 4. A linking member 54 is provided between the second handle member 30 and a latch housing 56, which houses a latch mechanism to engage the hemispherical end 22 of the inner shaft 12. The use of a linking member 54 enables relative rotational movement between the first and second handle parts 28,30 to be converted into linear movement of the latch housing 56.

The latch housing 56 contains the latch release member 40 which is connected to a latch member 58. The latch member 58 is biased into the position depicted in FIG. 4 by a resilient means, preferably a spring (not shown) and can be moved down within the latch housing 56 against the force of the resilient means. An upper surface of the latch member 58 is curved.

The handle is made of medical engineering plastics. Examples of suitable plastics include polyphenylsulfone (PPSU), such as that commercially available under the trade name Radel R from Solvay Advanced Polymers LLC, and polyetheretherketone (PEEK). The surgical instrument is made of medical grade alloys, such as aluminium alloys and steel alloys, covered with a insulated sleeve from a heat-shrunk polymer.

In use, a user first selects an appropriate surgical instrument 4 dependent upon the procedure being carried out. The surgical instrument 4 is then inserted into the handle 2, so that the inner and outer shafts 12, 14 enter the substantially cylindrical bore 39 in the handle 2. As the outer shaft enters the substantially cylindrical bore 39, the indentations 19 engage protrusions (not shown). The protrusions extend in the cylindrical bore 39 by a distance sufficient to engage the outer shaft 12 but not the inner shaft 14. The angled nature of the indentations 19 means they interact with the protrusions to align the instrument correctly, so that the flat surfaces 26 and bore 18 are correctly aligned with the second portion of the opening in the locking member 38.

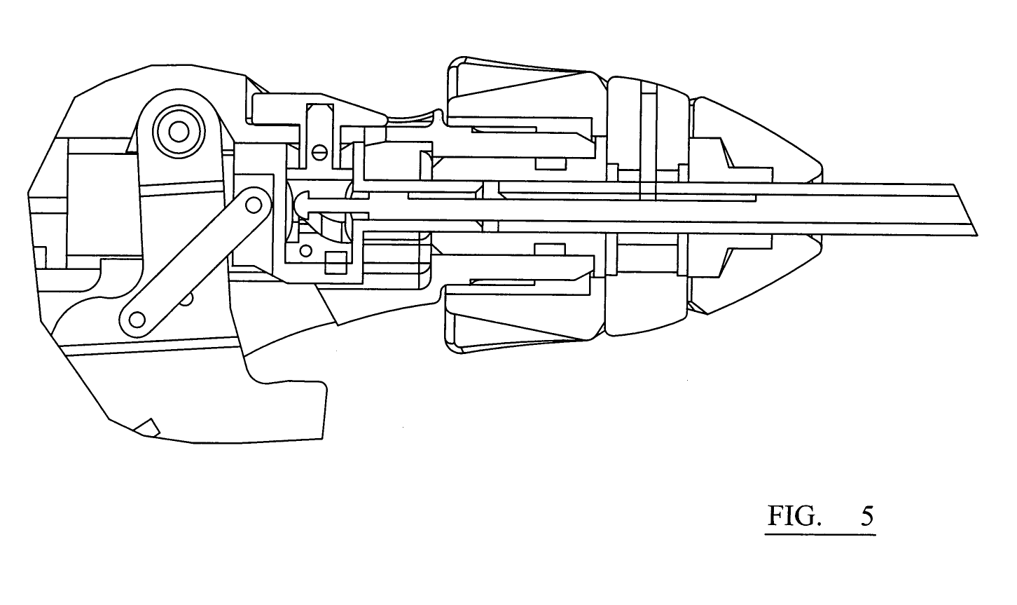

As the surgical instrument 4 is inserted further, the hemispherical end 22 engages the curved surface of the latch member 58, pushing the latch member down to the position depicted in FIG. 5. As the user continues to insert the surgical instrument 4, the hemispherical end 22 passes beyond the latch member 58 which moves upwards under the force of the resilient means. The hemispherical end 22 is now retained in the handle 2 by the action of the latch member 58 against the latch surface 26.

To lock the surgical instrument 4 against rotation relative to the handle, the locking member 38 is moved into the second position, so that the flat surfaces 16 are engaged by the second portion 44 of the opening. In addition the elongated member 46 engages the bore 18, preferably extending all the way through the bore 18 into the space provided by flattened portion 20.

The surgical instrument 4 is now locked in place within the handle by two mechanisms which effectively couple the outer shaft 12 to the first handle part 28 and the inner shaft 14 to the second handle part 30 (via latch housing 56 and link member 54). If the first and second handle parts 28,30 are rotated relative to each other the relative movement is transferred by the link member 54 to the latch housing 56, which moves within the first handle part 28 and hence moves the inner shaft 14 relative to the outer shaft 12, actuating the operative part of the surgical instrument 4 which in this case are scissors.

To remove the surgical instrument 4 from the handle 2 the locking member is moved into the first position, releasing the connection between the outer shaft 12 and the first handle part 28. Next, the latch release member 40 is pressed, moving the latch member 58 down so that it no longer engages the latch surface 26. The surgical instrument 4 can now be removed.

An alternate method (not illustrated) can also be used to insert the instrument into the handle. In this alternate method the user holds the handle in a closed position, so that the first and second handle parts 28,30 are close together. This moves the latch housing 56 towards the back of the handle.

Next, the surgical instrument 4 is inserted into the handle 2, so that the inner and outer shafts 12, 14 enter the substantially cylindrical bore 39 in the handle 2. As the outer shaft enters the substantially cylindrical bore 39, the indentations 19 engage protrusions (not shown). The protrusions extend in the cylindrical bore 39 by a distance sufficient to engage the outer shaft 12 but not the inner shaft 14. The angled nature of the indentations 19 means they interact with the protrusions to align the instrument correctly, so that the flat surfaces 26 and bore 18 are correctly aligned with the second portion of the opening in the locking member 38. The protrusions are positioned so that they hit the end of the indentations 19 when the outer shaft is in the correct position within the handle. The user continues to insert the surgical instrument 4 until this happens.

To lock the surgical instrument 4 against rotation relative to the handle, the locking member 38 is then moved into the second position, so that the flat surfaces 16 are engaged by the second portion 44 of the opening. In addition the elongated member 46 engages the bore 18, preferably extending all the way through the bore 18 into the space provided by flattened portion 20. The outer shaft is now secured in the handle but because the first and second handle parts 28,30 have been held closed, the inner shaft is not engaged with the latch. To engage the inner shaft the handle is opened. To assist this, the first and second handle parts 28, 30 may be biased into the open position by a resilient member, such as a spring. A user can then simply release the handle to engage the inner shaft.

The above described embodiment provides secure locking to the outer shaft in two ways, by the engagement of the second portion 44 of the locking member with the flat surfaces 16 and by the engagement of the elongated member 46 with the bore 18. It has been found that secure locking can still be achieved if only one of these is used.

In an alternate embodiment, the elongated member 46 is not included in the locking member 38 and the radial bore 18 in the outer shaft need not be provided. Otherwise the construction of this embodiment is the same as the above described first embodiment. The engagement of this embodiment with the outer shaft is depicted in FIGS. 7 and 8. As can be seen the locking member 60 includes a first portion 42 and second portion 44 as with the first embodiment. The outer shaft is held securely in place by the engagement of the second portion 44 with the flat surfaces 16 on the outer shaft, as can be seen depicted in FIG. 8.

This embodiment can also be used with a surgical instrument exactly the same as described for the first embodiment. Although the radial bore 18 will be present it does not have any effect on the engagement of the second portion 44 with the flat surfaces on the outer shaft.

In another alternate embodiment, which is the same as the first embodiment save as described below, a locking member 62 includes a single opening 64 which is slightly larger than the diameter of the outer shaft of the surgical instrument. The engagement of this embodiment with the outer shaft is depicted in FIGS. 9 and 10. The locking member 62 includes an elongated member 66 which extends partially into the opening 64 in a direction perpendicular to the longitudinal axis of the substantially cylindrical bore. In the surgical instrument, the outer shaft does not include flat surfaces 16, as shown by the circular cross section in FIGS. 9 and 10. However, the radial bore 18 is still provided.

As can best be understood with reference to FIGS. 9 and 10, the elongated member 66 extends a sufficient distance into the opening 64 that when the locking member 62 is moved into the second position, the elongated member extends into the bore 18. When the locking member 62 is in the first position, the elongated member does not extend far enough into the opening 64 to obstruct the insertion or removal of the surgical instrument into the cylindrical bore of the first handle part. The engagement of the elongated member 66 with the bore 18 in the outer shaft holds the outer shaft securely in place within the first handle part.

Thus, according to the invention a surgical instrument system is provided which is simple to manufacture and which can provide secure and reliable releasable attachment of a surgical instrument to a handle.

1. A surgical instrument for use in minimally invasive surgery; the instrument comprising:
an outer shaft having proximal and distal ends, a longitudinal axis and a through bore extending from the proximal end to the distal end;
an inner shaft having proximal and distal ends, positioned within the through bore and extending proximally beyond the proximal end of the outer shaft; and
an operative portion comprising a first part attached to the distal end of the outer shaft and a second part attached to the distal end of the inner shaft;
wherein a portion of the outer shaft towards the proximal end of the outer shaft has an external surface comprising two opposed flat surfaces which are substantially parallel to each other; and
wherein the proximal end of the inner shaft comprises an end portion with a maximum external dimension in a direction perpendicular to the longitudinal axis greater than the maximum external dimension of an adjoining portion of the inner shaft, and wherein a latch surface substantially perpendicular to the longitudinal axis extends between the end portion and the adjoining portion.

2. A surgical instrument according to claim 1, wherein the end portion is curved such that the external dimension of the end portion perpendicular to the longitudinal axis reduces towards the proximal end.

3. A surgical instrument according to claim 2, wherein the end portion is at least partially spherical.

4. (canceled)

5. (canceled)

6. (canceled)

7. (canceled)

8. A handle for use with a minimally invasive surgical instrument, the handle comprising:
a first handle part comprising a substantially cylindrical bore having a longitudinal axis for receiving a surgical instrument;
a second handle part attached to the first handle part for pivotal movement relative to the first handle part; and
wherein the first handle part further comprises a locking member extending through the substantially cylindrical bore that can be moved between first and second positions in a direction perpendicular to the longitudinal axis, wherein the locking member delimits an opening having a first portion with dimensions the same as or larger than the diameter of the substantially cylindrical bore and an adjoining second portion with at least one dimension smaller than the diameter of the substantially cylindrical bore, wherein in the first position the first portion is aligned with the substantially cylindrical bore and in the second position the second portion is aligned with the substantially cylindrical bore; and
wherein the first handle part further comprises a latch mechanism at the end of the substantially cylindrical bore.

9. A handle according to claim 8, wherein the latch mechanism comprises a latch member movable between first and second positions and wherein the latch member is biased in the first position by a resilient means, such that movement of the latch member into the second position is against a force exerted by the resilient means.

10. A handle according to claim 9, wherein the resilient means is a spring.

11. A handle according to claim 9, wherein the latch member comprises a surface angled relative to the longitudinal axis.

12. A handle according to claim 9, wherein the latch mechanism further comprises a release lever for moving the latch member into the second position.

13. (canceled)

14. A surgical instrument system for use in minimally invasive surgery, wherein the system comprises:
a surgical instrument comprising:
an outer shaft having proximal and distal ends, a longitudinal axis, and a through bore extending from the proximal end to the distal end;
an inner shaft having proximal and distal ends positioned within the through bore and extending proximally beyond the proximal end of the outer shaft; and
an operative portion comprising a first part attached to the distal end of the outer shaft and a second part attached to the distal end of the inner shaft; wherein
a portion of the outer shaft towards the proximal end of the outer shaft has an external surface comprising two opposed flat surfaces which are substantially parallel to each other; and
a handle comprising:
a first handle part comprising a substantially cylindrical bore for receiving a part of the outer shaft and the inner shaft;
a second handle part attached to the first handle part for pivotal movement relative to the first handle part; and wherein
the first handle part further comprises a locking member extending through the substantially cylindrical bore that can be moved between first and second positions in a direction perpendicular to the longitudinal axis, wherein the locking member delimits an opening having a first portion with dimensions the same as or larger than the diameter of the substantially cylindrical bore and an adjoining second portion with a dimension corresponding to the distance between the two opposed flat surfaces, wherein in the first position the first portion is aligned with the substantially cylindrical bore and in the second position the second portion is aligned with the substantially cylindrical bore such that when the proximal end of the surgical instrument is inserted into the substantially cylindrical bore and the locking member is moved into the second position, the two opposed flat surfaces are engaged by the second portion;
wherein the proximal end of the inner shaft of the surgical instrument comprises an end portion with a maximum external dimension in a direction perpendicular to the longitudinal axis greater than the maximum external dimension of an adjoining portion of the inner shaft, wherein a latch surface substantially perpendicular to the longitudinal axis extends between the end portion and the adjoining portion; and wherein the first handle part further comprises a latch mechanism at the end of the substantially cylindrical bore for engaging the latch surface.

15. (canceled)

16. (canceled)

17. (canceled)

18. (canceled)

19. (canceled)

20. (canceled)
